(19) United States
(12) Patent Application Publication
(10) Pub. No.: US 2005/0055075 A1
(43) Pub. Date: Mar. 10, 2005

(54) Methods for the manufacture of porous prostheses

(76) Inventors: Leonard Pinchuk, Miami, FL (US); Yasushi P. Kato, Pembroke Pines, FL (US)

Correspondence Address: KRAMER LEVIN NAFTALIS & FRANKEL LLP;INTELLECTUAL PROPERTY DEPARTMENT, 919 THIRD AVENUE, NEW YORK, NY, 10022, US

(21) Appl. No.: 10/657,544

(22) Filed: Sep. 8, 2003

(51) Int. Cl. A61F 2/06

(52) U.S. Cl. 623/1.1; 264/41

(57) ABSTRACT

A method for the manufacture of a biocompatible porous prosthesis comprising the steps of applying a solution to a porous support structure for a prosthesis, said solution comprising a biocompatible block copolymer including one or more elastomeric blocks and one or more thermoplastic blocks, and a first solvent capable of dissolving said copolymer; and applying a second solvent capable of dissolving said first solvent but incapable of dissolving said copolymer to the surfaces of said prosthesis and thereby causing said copolymer to precipitate onto said support structure.

### FIELD OF THE INVENTION

The present invention relates to porous vascular prostheses and methods for making them. More specifically the present invention relates to porous vascular grafts, patches, stents, stent-grafts and the like comprising a porous polystyrene-polyisobutylene-polystyrene triblock polymer produced by a phase inversion technique.

### BACKGROUND OF THE INVENTION

Medical prostheses for implantation into the body are well known in the art. It is desirable that such prostheses be stable for the duration of the lifetime of the recipient and that they be made of materials which are biocompatible. Implantable prostheses must be formed in a manner to substantially prevent their cracking, crazing or degradation in the body. Implantable prostheses, in particular vascular prostheses having porous structures, are important for use in blood filters, blood vessels and other devices.

Implantable prostheses include implantable medical devices such as vascular grafts, endoluminal grafts, hernia patches, vascular patches, intraocular lenses, glaucoma tubes and anchoring means, finger joints, indwelling catheters, pacemaker lead insulators, breast implants, heart valves, knee and hip joints, vertebral disks, meniscuses, tooth liners, plastic surgery implants, tissue expanders, drug release membranes, subcutaneous ports, injection septums and the like. Vascular prostheses include but are not limited to vascular grafts, vascular patches, stents, stent-grafts, vascular access grafts, suture rings, and the like.

Porous vascular prostheses, used in blood vessels greater than 8 mm, have been used for over 30 years. These vascular grafts are usually made from knitted or woven Dacron (polyester terephthalate or PET), a non-elastomeric polymer which performs well in the body.

Smaller caliber vascular prostheses are also known, and include those made from expanded polytetrafluoroethylene, or ePTFE, a non-elastomeric polymer. These grafts are manufactured by many companies, including Gore, Impra, Boston Scientific, Edwards, Bard and the like. The most common size for these grafts are 6 mm in diameter.

Porosity is usually required in vascular prosthesis for three reasons: (1) to heal the anastomosis, i.e., to facilitate tissue growth from the natural artery into the pores of the prosthesis to essentially heal the prosthesis and prevent leakage at the anastomoses; (2) to permit the tissue to grow through the wall of the prosthesis and line the inside lumen of the prosthesis and essentially render it biocompatible; and (3) to provide porosity or texturing on the inside (luminal side) of the prosthesis to help stick a neointima to the prosthesis thereby rendering the prosthesis hemocompatible.

There have been many attempts to manufacture vascular grafts using elastomers under the premise that an elastomeric material will emulate the natural compliance of normal blood vessels and allow prostheses to be made that are functional at diameters less than 6 mm (e.g., 4 and 3 mm diameters). Further, elastomeric materials are known to have the intrinsic ability to seal around suture holes at the anastomosis or when punctured by dialysis needles.

Elastomers that have been typically used in the development and research of compliant vascular prostheses are polyurethanes and silicone rubbers. Polyurethanes, however, are known to materially degrade with time in the body. L. Pinchuk, A Review of the Biostability and Carcinogenity of Polyurethanes in Medicine and the New Generation of “Biostable” Polyurethanes, J. Biomaterial Science, Polymer Ed., Vol 6, No.3, pp 225-267 (1994). Many of the elastomeric vascular prostheses made from polyurethane fail due to biodegradation and aneurysm formation. In addition, polyurethane elicits an inflammatory reaction that renders the prosthesis difficult to remain patent at small diameters (i.e., less than 6 mm). Silicone rubber has also been used as a material for vascular grafts (Possis, Inc.); however, it has poor blood compatibility, is difficult to process due to its thermoset nature and is rarely used at the present time.

Pinchuk, U.S. Pat. Nos. 5,741,331 and 6,102,933, incorporated herein by reference, describe the use of copolymers in the manufacture of implantable or insertable medical devices. The copolymers used include a polyolefinic elastomeric triblock star or linear copolymer where the backbone comprises alternating units of quaternary and secondary carbons. Prostheses made of such materials do not crack or degrade even after substantial periods of use. A triblock polymer referred to as polystyrene-polyisobutylene-polystyrene, (also referred to as poly(styrene-isobutylene-styrene)) (“SIBS”) is a preferred class of elastomeric material for the formation of compliant vascular prostheses.

Methods for coating a medical device, such as a stent, are described in Pinchuk, U.S. Pat. No. 6,545,097, the subject matter of which is incorporated herein by reference. This method is a solvent-based technique wherein a solution containing dissolved copolymer is sprayed upon a porous prosthesis (e.g., catheter, catheter balloon, stent, stent graft, vascular graft, etc.) Other methods for forming porous elastomeric vascular prostheses are described in Dereume et al., U.S. Pat. No. 6,309,413 and MacGregor, U.S. Pat. No. 4,936,317.

### OBJECTS OF THE INVENTION

It is a primary object of the invention to provide improved porous prostheses made of elastomeric materials which are biocompatible.

It is a further object of the invention to provide such prostheses having carefully controlled porosity characteristics.

Yet another object of the present invention is to provide methods for preparing porous vascular prostheses, which are cost effective, efficient and technically reliable.

A still further object of the invention is to provide a method for preparing porous vascular prostheses, with or without a porous support structure, comprising SIBS.

### SUMMARY OF THE INVENTION

These and other objects, features and advantages are achieved by making a coated prosthesis by the methods of the invention which comprise the steps of: (a) applying a solution comprising (i) a biocompatible block polymer including one or more elastomeric blocks and one or more thermoplastic blocks and (ii) a first solvent capable of dissolving the biocompatible block polymer, to a porous support structure for a prosthesis; and (b) applying a second solvent capable of dissolving the first solvent but not capable of dissolving the biocompatible block polymer to the coated support structure and thereby causing said copolymer to precipitate onto said porous support structure. Neither the first nor the second solvent is capable of dissolving the porous support structure.

These and other objects, features and advantages of the invention are also achieved in methods for producing self-supporting prostheses by depositing the solution of block polymer and first solvent on a mandril for a prosthesis; applying the second solvent to the coated mandril, thereby causing said copolymer to precipitate onto said mandril; removing solvent from the copolymer deposited on the mandril; and removing the so-formed porous prosthesis from the mandril.

In preferred aspects of the invention, the biocompatible block copolymer comprises isobutylene and styrene or α-methylstyrene. A porous support structure or a mandril for a prosthesis is submerged in a solution of polymer in a suitable solvent. The wetted support structure is then submerged in a second solvent capable of dissolving the first solvent but not capable of dissolving the block copolymer, thereby causing the copolymer to precipitate out on the porous support or on the mandril. The coated support structure or the so-formed porous prosthesis on the mandril is then dried by removing residual solvent.

In another aspect of the invention, a porous support structure or a mandril for a prosthesis is coated with a biocompatible copolymer in a method comprising the steps of: (a) forming a solution comprising (i) a biocompatible block copolymer and (ii) a mixture of solvents comprising a first solvent capable of dissolving said copolymer and a second solvent capable of dissolving said first solvent but not capable of dissolving said copolymer, said second solvent having a boiling point higher than said first solvent and being present in an amount less than that which causes said copolymer to precipitate out of said first solvent; (b) submerging a porous support structure or mandril for a prosthesis in the solution formed in step (a); (c) volatilizing said first solvent from said solution, thereby causing said copolymer to precipitate onto said support structure or mandril; and (d) removing said second solvent from the coated support structure or the so-formed porous prosthesis.

In still another embodiment the method for the manufacture of a biocompatible porous prosthesis comprises the steps of: forming a solution comprising a biocompatible block copolymer including one or more elastomeric blocks and one or more thermoplastic blocks, and a first solvent capable of dissolving said copolymer; pouring said solution into a mold; chilling said solution to form a gel; removing said gel from said mold and immersing it in a second solvent capable of dissolving said first solvent but incapable of dissolving said copolymer; and heating the gel and second solvent and thereby causing the block copolymer to precipitate and form a porous solid having interconnecting pores. Alternatively, the phase inversion process using two solvents of different boiling points can be used by pouring the solution comprising the block polymer and the mixture of solvents into the mold, chilling the solution to form a gel, removing the gel and heating the gel to flash off the lower-boiling solvent.

### BRIEF DESCRIPTION OF THE DRAWINGS

FIG. 1 is a (60×) scanning electron microscope (“SEM”) micrograph of a PET support structure prior to coating with porous copolymer.

FIG. 2 is an SEM micrograph of a porous SIBS-coated support structure wherein the pores are approximately 1 mm in diameter.

FIG. 3 is porous SIBS-coated support structure wherein the pores are approximately 1 mm in diameter.

FIG. 4 is a graph showing the permeability of the copolymer as a function of the solids content of copolymer in the solution prior to precipitation using one or two dips in which the second solvent is present in an amount 5% below the titration point.

FIG. 5 is a graph showing the permeability of the copolymer as a function of the solids content of copolymer in the solution prior to precipitation using one or two dips, wherein the second solvent is present in an amount 50% below the titration point.

### DETAILED DESCRIPTION OF THE PREFERRED EMBODIMENTS

The present invention relates to the manufacture of porous prostheses using biocompatible block copolymers. The copolymers are deposited by phase inversion techniques and are porous. They may be deposited on porous support structures or on a mandril for a prosthesis.

Prostheses with Porous Support Structures

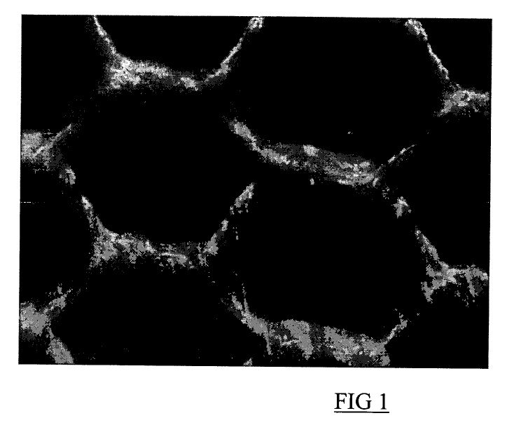

Porous support structures are those structures that will allow blood to leak and tissue to ingrow through the pores of the structure. The pore size allowing tissue ingrowth and blood leakage can range from 1 micron in diameter to many millimeters in diameter. Support structures include PET weaves, knits, mats, non-weaves and braids used for vascular grafts, vascular patches, stent-grafts, blood filters, protection devices, embolizing particles, and the like. Patches can be used to close hernias as well as arteries following dissection, such as the carotid artery following endarterectomy. FIG. 1 shows an example of a knitted porous structure that can be used in the methods of the invention.

The support structure can also be a stent, such as the Wallstent (Boston Scientific) where the braid pattern of the wire stent provides a mesh with pores in the range of 1 to 10 mm². In this stent embodiment, the viscosity of the coating solution is adjusted so that when the stent is dipped and removed from the solution of copolymer, solution bridges the interstices of the stent, and, when dry, provides a porous membrane covering these interstices. Coating a stent in this manner essentially provides a stent-graft. The porous structure can be built up sequentially by dipping the coated stent one or more times in the same or a different solution.

Self-Supporting Prostheses

In an alternate embodiment, prostheses without a support structure can be produced using a mandril, e.g. a rod-shaped mandril or a mold. The mandril, like the porous support structure, is dipped into the phase inverting solution, removed and dried to form a porous film over the mandril. The porous structure can be built up sequentially by dipping the mandril one or more times in the same or a different solution of copolymer. After the porous structure is dried, it can be removed from the mandril to form, for example, a porous tube.

Block Copolymers

Block copolymers suitable for the practice of the present invention preferably have a first elastomeric block and a second thermoplastic block. A block may be a monomer, dimer, oligomer, or any other polymeric unit. More preferably, the block copolymers have a central elastomeric block and thermoplastic end blocks. Even more preferably, such block copolymers have the general structure:
(a) BAB or ABA (linear triblock),
(b) B(AB)ₙ or A(BA)ₙ (linear alternating block), or
(c) X-(AB)ₙ or X-(BA)ₙ (includes diblock, triblock and other radial block copolymers),
   where A is an elastomeric block, B is a thermoplastic block, n is a positive whole number and X is a starting seed molecule.

Most preferred are X-(AB)ₙ structures, which are frequently referred to as diblock copolymers and triblock copolymers where n=1 and n=2, respectively (this terminology disregards the presence of the starting seed molecule, for example, treating A-X-A as a single A block with the triblock therefore denoted as BAB). Where n=3 or more, these structures are commonly referred to as star-shaped block copolymers.

The A blocks are preferably soft elastomeric components which are based upon one or more polyolefins, more preferably a polyolefinic block having alternating quaternary and secondary carbons of the general formulation: —(CRR′—CH2)ₙ—, where R and R′ are linear or branched aliphatic groups such as methyl, ethyl, propyl, isopropyl, butyl, isobutyl and so forth, or cyclic aliphatic groups such as cyclohexane, cyclopentane, and the like, with and without pendant groups. Polymers of isobutylene, 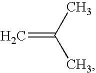
(i.e., polymers where R and R′ are the same and are methyl groups) are most preferred.

The B blocks are preferably hard thermoplastic blocks that, when combined with the soft A blocks, are capable of, inter alia, altering or adjusting the hardness of the resulting copolymer to achieve a desired combination of qualities. Preferred B blocks are polymers of methacrylates or polymers of vinyl aromatics. More preferred B blocks are (a) made from monomers of styrene 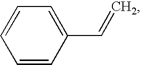
styrene derivatives (e.g., alpha-methylstyrene, ring-alkylated styrenes or ring-halogenated styrenes) or mixtures of the same or are (b) made from monomers of methylmethacrylate, ethylmethacrylate, butylmethacrylate, hydroxyethyl methacrylate or mixtures of the same.

The properties of the block copolymers used in connection with the present invention will depend upon the lengths of the A blocks and B blocks, as well as the relative amounts of each. For example, the elastomeric properties of the block copolymer will depend on the length of the A block chains, with a weight average molecular weight of from about 2,000 to about 30,000 Daltons tending to produce rather inelastic products, and a weight average molecular weight of 40,000 Daltons or above tending to produce products that are more soft and rubbery. Hence, for purposes of the present invention, the combined molecular weight of the block copolymer is preferably in excess of 40,000 Daltons, more preferably in excess of 60,000 Daltons, and most preferably between about 60,000 to about 300,000 Daltons.

As another example, the hardness of the block copolymer is proportional to the relative amount of B blocks. In general, the copolymer has a preferred hardness that is between about Shore 20A and Shore 75D, and more preferably between about Shore 40A and Shore 90A. This result can be achieved by varying the proportions of the A and B blocks, with a lower relative proportion of B blocks resulting in a copolymer of lower hardness, and a higher relative proportion of B blocks resulting in a copolymer of higher hardness. As a specific example, high molecular weight (i.e., greater than 100,000 Daltons) polyisobutylene is a soft gummy material with a Shore hardness of approximately 10A. Polystyrene is much harder, typically having a Shore hardness on the order of 100D. As a result, when blocks of polyisobutylene and styrene are combined, the resulting copolymer can have a range of hardnesses from as soft as Shore 10A to as hard as Shore 100D, depending upon the relative amounts of polystyrene and polyisobutylene. In general, to achieve a preferred hardness ranging from Shore 30A to Shore 90A, the amount of polystyrene ranges from between 2 and 25 mol %. More preferably, the preferred hardness ranges from Shore 35A to Shore 70A and the amount of polystyrene ranges from 5 to 24 mol %.

Polydispersity (i.e., the ratio of weight average molecular weight to number average molecular weight) gives an indication of the molecular weight distribution of the copolymer, with values significantly greater than 4 indicating a broad molecular weight distribution. The polydispersity has a value of one when all molecules within a sample are the same size. Typically, the copolymers for use in connection with the present invention have a relatively tight molecular weight distribution, with a polydispersity of about 1.1 to 1.7.

One advantage associated with the above-described copolymers is their relatively high tensile strength. For example, the tensile strength of triblock copolymers of polystyrene-polyisobutylene-polystyrene frequently ranges from 2,000 to 4,000 psi or more.

Another advantage of such copolymers is their resistance to cracking and other forms of degradation under in vivo conditions. In addition, these polymers exhibit excellent biocompatibility, including vascular compatibility, as demonstrated by their tendency to provoke minimal adverse tissue reactions as demonstrated by reduced polymorphonuclear leukocyte and reduced macrophage activity. Still further, these polymers are generally hemocompatible as demonstrated by their ability to minimize thrombotic occlusion of small vessels as demonstrated by coating such copolymers on coronary stents.

Preparation of Block Copolymers

The above-described block copolymers can be synthesized using any appropriate method known in the art. A preferred process of making the block copolymers is by carbocationic polymerization involving an initial polymerization of a monomer or mixtures of monomers to form the A blocks, followed by the subsequent addition of a monomer or a mixture of monomers capable of forming the B blocks.

Such polymerization reactions can be found, for example, in additional U.S. Pat. Nos. 4,276,394, 4,316,973, 4,342,849, 4,910,321, 4,929,683, 4,946,899, 5,066,730, 5,122,572 and/or Re. 34,640. Each of these patents is hereby incorporated by reference in its entirety.

The techniques disclosed in these patents generally involve a “catalyst starting molecule” (also referred to as “initiators”, “telechelic starting molecules”, “seed molecules” or “inifers”), which can be used to create X-(AB)ₙ structures, where X is the catalyst starting molecule, and n can be 1, 2, 3 or more. As noted above, the resulting molecules are referred to as diblock copolymers where n is 1, triblock copolymers (disregarding the presence of the starting molecule) where n is 2, and star-shaped block copolymers where n is 3 or more.

In general, the polymerization reaction is conducted under conditions that minimize or avoid chain transfer and termination of the growing polymer chains. Steps are taken to keep active hydrogen atoms (water, alcohol and the like) to a minimum. The temperature for the polymerization is usually between −10° and −90° C., the preferred range being between −60° and −90° C., although lower temperatures may be employed if desired.

Preferably, one or more A blocks, for example, polyisobutylene blocks, are formed in a first step, followed by the addition of B blocks, for example, polystyrene blocks, at the ends of the A blocks.

More particularly, the first polymerization step is generally carried out in an appropriate solvent system, typically a mixture of polar and non-polar solvents such as methyl chloride and hexanes. The reaction bath typically contains: the aforementioned solvent system, olefin monomer, such as isobutylene, an initiator (inifer or seed molecule) such as tert-ester, tert-ether, tert-hydroxyl or tert-halogen containing compounds, and more typically cumyl esters of hydrocarbon acids, alkyl cumyl ethers, cumyl halides and cumyl hydroxyl compounds as well as hindered versions of the above, a coinitiator, typically a Lewis Acid, such as boron trichloride or titanium tetrachloride.

Electron pair donors such as dimethyl acetamide, dimethyl sulfoxide, or dimethyl phthalate can be added to the solvent system. Additionally, proton-scavengers that scavenge water, such as 2,6-di-tert-butylpyridine, 4-methyl-2,6-di-tert-butylpyridine, 1,8-bis(dimethylamino)-naphthalene, or diisopropylethyl amine can be added.

The reaction is commenced by removing the tert-ester, tert-ether, tert-hydroxyl or tert-halogen (herein called the “tert-leaving groups”) from the seed molecule by reacting it with the Lewis acid. In place of the tert-leaving groups is a quasi-stable or “living” cation which is stabilized by the surrounding tertiary carbons as well as the polar solvent system and electron pair donors. After obtaining the cation, the A block monomer, such as isobutylene, is introduced which cationically propagates or polymerizes from each cation on the seed molecule. When the A block is polymerized, the propagated cations remain on the ends of the A blocks. The B block monomer, such as styrene, is then introduced which polymerizes and propagates from the ends of the A block. Once the B blocks are polymerized, the reaction is terminated by adding a termination molecule such as methanol, water and the like.

As is normally the case, product molecular weights are determined by reaction time, reaction temperature, the nature and concentration of the reactants, and so forth. Consequently, different reaction conditions will produce different products. In general, synthesis of the desired reaction product is achieved by an iterative process in which the course of the reaction is monitored by the examination of samples taken periodically during the reaction—a technique widely employed in the art. To achieve the desired product, an additional reaction may be required in which reaction time and temperature, reactant concentration, and so forth are changed.

Additional details regarding cationic processes for making copolymers are found, for example, in U.S. Pat. Nos. 4,276,394, 4,316,973, 4,342,849, 4,910,321, 4,929,683, 4,946,899, 5,066,730, 5,122,572 and/or Re. 34,640.

The block copolymers described in the preceding paragraphs may be recovered from the reaction mixtures by any of the usual techniques including evaporation of solvent, precipitation with a non-solvent such as an alcohol or alcohol/acetone mixture, followed by drying, and so forth. In addition, purification of the copolymer can be performed by sequential extraction in aqueous media, both with and without the presence of various alcohols, ethers and ketones.

Methods for Preparing Biocompatible Prostheses

Biocompatible porous prostheses are made using solvent-based techniques in which the block copolymer is dissolved in a solvent and the block-copolymer solution is then applied to a porous support structure, for example a porous vascular prosthesis, or to a mandril for a porous prosthesis. A second solvent, capable of dissolving the first solvent but incapable of dissolving the block copolymer, is then applied to the surfaces of the support structure or mandril, causing the block copolymer to precipitate thereon.

In preferred embodiments, the copolymer comprises isobutylene and styrene or α-methylstyrene. A porous support structure or mandril for a prosthesis is submerged in a solution containing copolymer. After the first submersion, the wetted porous support structure or mandril is submerged in a second solvent that is capable of dissolving the first solvent but not the copolymer, thereby causing the block copolymer to precipitate onto the surfaces of the support structure or mandril. The remaining first and second solvent is then removed from the structure by heating the coated structure or mandril.

Alternatively, a biocompatible porous prosthesis can be made using solvent-based techniques in which the block copolymer is dissolved in a mixture of solvents and subsequently applied to a porous support structure or mandril. The mixture of solvents comprises a first solvent which is capable of dissolving the block copolymer and a second solvent which is not incapable of dissolving the block copolymer but is capable of dissolving the first solvent. The second solvent has a boiling point higher than that of the first solvent and is present in an amount less than that which causes the block copolymer to precipitate out of the first solvent. The ratio of poor solvent to good solvent that causes the block copolymer to precipitate out is denoted as the “titration point”. The solution is then heated so that the first solvent is flashed off. The block copolymer is precipitated onto the porous support structure or mandril. In preferred embodiments, the block copolymer comprises polyisobutylene and polystyrene or poly(α-methylstyrene). The second solvent is preferably present in an amount not more than 99% of the amount which would cause the copolymer to precipitate.

The Solvents and Copolymer Solutions

Suitable first solvents are generally non-polar solvents. Typical examples of non-polar solvents include, but are not limited to, toluene, hexanes, heptanes, tetrahydrofuran, cyclohexane, methyl cyclohexane and the like. The biocompatible block copolymer is dissolved in the first solvent. Broadly, the solutions of copolymer contain 0.5% to 50% by weight copolymer by weight of solution and preferably from 7% to 15%, as measured before introduction of the second solvent.

Suitable solvents that are capable of dissolving the first solvent but are not capable of dissolving the block copolymer include methanol, propanol, 2-propanol, ethanol, 1-butanol, 2-butanol, acetone, hexanol, and the like. The first solvent and the second solvent must be cosoluble, i.e., the first solvent must dissolve the block copolymer while the second solvent must not dissolve the block copolymer but must be soluble in the first solvent.

Techniques of Application

In a preferred embodiment, the copolymer solution is applied to the support structure or mandril by dipping a mesh, a stent, a frame, a mandril or the like, into a solution of copolymer dissolved in a compatible solvent. The dipped support structure or mandril is then removed from the copolymer solution, and, while wet, submerged in a different, second solvent. The second solvent is capable of dissolving the first solvent, but is not capable of dissolving the copolymer. Because the block copolymer is insoluble in the second solvent and the first solvent is soluble in the second, the first solvent will diffuse into the second solvent and the copolymer will precipitate. The copolymer forms a porous network film in the area of the support structure or mandril dipped into the solution of copolymer. This process is called phase inversion and is known in the art. The coated structure or coated mandril is then heated to drive off residual solvent.

In an alternative embodiment, the solution into which the support structure or mandril is dipped comprises the block copolymer, a first solvent and a second solvent. The copolymer is soluble in the first solvent but not in the second solvent. The first solvent has a lower boiling point than the second solvent. A solution comprising the block copolymer and the first solvent is titrated with the second solvent to the point where the copolymer precipitates. The amount of second solvent necessary to precipitate the copolymer from the solution is noted and is called the “titration point”. A fresh solution of block copolymer in first solvent is then prepared. The second solvent is added to that solution in preferably 90% to 95% of the titration point. The support structure or mandril is then dipped into this solution, removed and then heated to a temperature above the boiling point of the first solvent but below the boiling point of the second solvent. This causes the first solvent to be flashed off, leaving the block copolymer in the second solvent where it then precipitates. Further heating of the block copolymer and second solvent causes the second solvent to flash off, leaving the porous copolymer precipitated on the support structure or mandril.

As those well versed in the art will appreciate, the solubility of a polymer in a solvent is dependant upon temperature and thus heating the solvent or the polymer solution will help solubilize the polymer. Some polymers in some solvents form a swollen gel rather than a true liquid solution. Heating such gels can change them into true liquid solutions. Conversely, if a polymer is soluble in a solvent at room temperature and the polymer solution is chilled, the polymer system can be converted to a gel. This temperature effect can be used to make thick coatings of the polymer solution on the porous support or mandril, as the case may be, as described below.

The block copolymer is dissolved in a first solvent, similar to those first solvents described above, and the solution poured into a mold and chilled. The polymer system thus forms a gel. The gel is then removed from the mold as a quasi-solid gelled structure and immersed in a second solvent which is a good solvent for the first solvent but a poor solvent for the copolymer. The second solvent dissolves the first solvent and thereby replaces it. As the mass returns to room temperature, it precipitates into a porous network of interconnecting pores. Thick layers of porous structures can be made in this manner.

Thick gels as described can also be used for vascular access grafts where thick elastomers are required to seal the graft following removal of dialysis needles. It can also be appreciated that the solvent system can be comprised of a first good solvent and a second poor solvent with concentrations below the titration point.

While the phase inversion method is advantageously performed by dipping the support structure or mandril in a solution, as described, these solutions can also be applied by solvent casting, spin coating, web coating, solvent spraying, ink jet printing and combinations of these processes. If desired, for example, to achieve a desired coating thickness, such coating techniques can be repeated or combined to build up the coated layer to the desired thickness. Coating thickness can be varied in other ways as well. For example, coating thickness can be increased by modification of the coating process parameters in solvent spraying, such as increasing flow rate, decreasing solids content, slowing the movement of the device to be coated relative to the spray nozzle, providing repeated passes, decreasing the temperature and so forth.

Control over the porosity of the prosthesis can be attained by adjustment of the solvents, copolymer concentration, viscosity of solutions, temperature, etc. Further, layers of copolymer with different porosity can be built up on the support structure or mandril using different chemistries. For example, if a porous structure is desired with a gradient of increasingly larger pores on either side of the support structure, the support structure can be dipped sequentially into solutions with less and less polymer solids to provide larger pore sizes. Likewise, where a mandril is dipped into the solvent system, removed and phase inverted as described above, the process may be repeated to build up a desired thickness of porous copolymer, with or without a gradient. After drying the so-formed, self-supporting porous prosthesis, is removed from the mandril.

Although it is technically feasible to measure the pore size of the copolymer deposited by the phase inversion methods of the invention using instrumentation such as an electron microscope, it is impractical as the pores interconnect throughout the thickness of the device and it is cumbersome to determine an actual pore size. A more practical method of measuring “pore size”, although less direct, is to measure the permeability of the material. The permeability is measured in units of mL/cm².min. For vascular devices, a membrane, such as the phase inverted structures described herein, is clamped between two circular channels of 1 cm² crossectional area. Fluid at physiological pressure, usually 150 mmHg, is flowed through one channel, through the membrane and out the other channel. The fluid is collected over a 1 minute period and the amount of fluid recorded. By way of example, if a phase inverted membrane is clamped between the channels and 200 mL of fluid at 150 mmHg pressure is flowed through the membrane, the permeability is 200 mL/cm².min. If a different membrane is used having a smaller pore size, it would be expected that the permeability would be less than 200 mL/cm².min. In this manner, the “effective” pore size can be determined.

It is important that the permeability be approximately zero (0) at 150 mmHg pressure for vascular grafts. At zero permeability, blood will not leak through the wall of the vessel. However, the graft must also be sufficiently permeable to allow tissue ingrowth. Different materials having different surface tension properties will display different permeability characteristics with the same fluid. Highly hydrophobic materials such as SIBS may be porous yet may not permit permeation by water at physiological pressures. However, when pressures are increased substantially, these same porous membranes will become permeable to water.

### EXAMPLE 1

### Block Copolymer Synthesis

A polystyrene-polyisobutylene-polystyrene block copolymer is synthesized using known techniques. As is well known by those versed in the art of cationic chemistry, all solvents and reactants must be moisture, acid and inhibitor-free. Therefore, it may be necessary, depending upon the grade of material purchased, to distill these chemicals or flow them through columns containing drying agents, inhibitor removers and the like, prior to introducing them into the reaction procedure.

Assuming that all solvents are pure and moisture- and inhibitor-free, styrene is added to a dried, airtight styrene mixing tank. The tank is initially chilled to between −19° C. (the condensation point of methyl chloride) and −31 ° C. (the freezing point of pure styrene) using liquid nitrogen or other heat transfer media, whereupon methyl chloride gas is condensed and added. Next, di tert-butyl-pyridine is mixed with hexanes and added to the styrene tank, followed by flushing with further hexanes. A small amount of isobutylene is then added to the styrene tank, followed by sufficient hexanes to bring the total hexane weight in the styrene mixing tank to the desired amount. The temperature is then brought to about −80° C. and maintained at that temperature until used.

Hexanes are discharged into a dried, airtight reactor, containing cooling coils and a cooling jacket. The reactor with the hexanes is cooled with liquid nitrogen or other heat transfer media. Methyl chloride is condensed into the reactor by bubbling the gas through the cooled hexanes. A hindered t-butyl dicumyl ether, dimethyl phthalate and di tert-butyl-pyridine are added to the reactor, flushing with hexanes. Isobutylene is charged and condensed into the reactor by bubbling the gas thought the cooled solvent system. The temperature is maintained at about −80° C. After the isobutylene is added to the reactor, titanium tetrachloride is then charged to the reactor, flushing with hexanes, to start the reaction. After the appropriate amount of isobutylene has been added, the reaction is allowed to continue for 15 to 30 min. The contents of the styrene tank (prechilled to −60 to −80° C.) are then added to the reactor, maintaining the reactor at a temperature of about −80° C. After adding all the contents of the styrene tank, the contents of the reactor are allowed to react an additional 15 to 45 minutes, with samples withdrawn periodically and analyzed by FTIR or other means to determine the styrene content in the copolymer. Once the styrene content is reached, the reaction is quenched with methanol.

The reactor is then allowed to warm to room temperature, while monitoring any pressure increases, and the methyl chloride is removed from the reactor by boiling it and condensing it into a chilled collection tank. An additional amount of hexanes, or other solvent, such as tetrahydrofuran or toluene is added to the reactor to replace the removed methyl chloride. These additional solvents are used to solubilize the polymer to enable it to be drained out of the reactor, as otherwise the polymer becomes too thick to readily flow. The copolymer solution from the reactor is then precipitated in methanol in an amount equal to the amount of initial copolymer and hexanes to be coagulated. The precipitated polymer is then poured into a sieve, the polymer removed and dried in a vacuum oven for at least 24 hours at approximately 125° C. under full vacuum.

### EXAMPLE 2

### Solvent Based Method of Coating a Vascular Stent

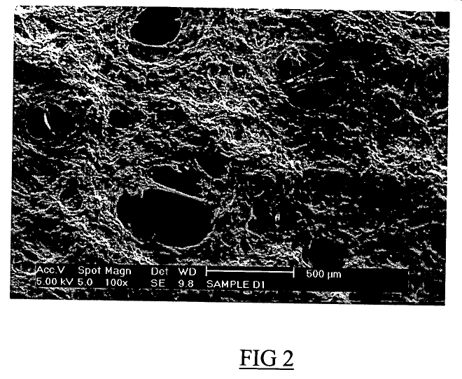

A solution containing 5 grams of polystyrene-polyisobutylene-polystyrene block polymer (SIBS) such as that described in Example 1 is dissolved in 95 grams of toluene, to provide a block polymer content of 5%. A vascular graft, 6 mm diameter polyethylene terephthalate (PET) (Dacronwoven tube, such as those marketed by Boston Scientific under the Trade Name “Hemashield Graft”) is dipped in the solution and slowly withdrawn. The wet tube is then submerged in a second solvent, 2-propanol, for 1 hour. The toluene dissolves in the 2-propanol and the SIBS precipitates to form a porous white network that is well adhered to the PET mesh tube. The coated PET tube is dried in an oven at 70° C. to 80° C. for 1 hour to remove the 2-propanol. A porous, SIBS, vascular graft, reinforced with PET, is formed. FIG. 2 shows a porous structure made in this manner.

### EXAMPLE 3

### Solvent-Based Method of Coating a Vascular Stent Graft

20 grams of SIBS are dissolved in 80 grams of toluene to provide a SIBS content of 20%. A braided wire stent, such as theWallstent (Boston Scientific) is submerged into this SIBS solution and then slowly removed so that the SIBS wicks across the interstices of the stent. The wet Wallstent is then submerged in 2-propanol and soaked for 1 hour at 50° C. The toluene dissolves in the 2-propanol and the SIBS precipitates in place to form a porous white network. The coated Wallstent is then dried in an oven at 70° C. for 1 hour to remove the 2-propanol. The process is repeated to provide a thicker layer of SIBS on the Wallstent. A porous SIBS stent-graft is formed in this manner.

### EXAMPLE 4

### Solvent-Based Method of Coating a Vascular Stent-Graft

20 grams of SIBS are dissolved in 80 grams of hexane to provide a SIBS content of 20%. A 20 cm long 8 mm diameter Wallstent (Boston Scientific) is dipped into the SIBS solution so that only 15 cm of the stent are in contact with the solution. The Wallstent is then slowly withdrawn so that the SIBS wicks across the interstices of the stent. 5 cm of the wetted Wallstent are then submerged in methanol causing the hexane in the solution wetting that section to dissolve in the methanol, i.e. to phase invert, and causing the SIBS to precipitate on the Wallstent and render the 5 cm end porous. The entire Wallstent is dried in an oven at 70° C. for 30 minutes to remove the methanol and the hexane from the 5 cm end of the Wallstent and the hexane from the middle 10 cm. The middle 10 cm section dries non-porous. The remaining bare 5 cm end of the Wallstent is then dipped in the SIBS solution and then in methanol and dried as above. The result is a stent-graft comprised of SIBS where 5 cm on both ends are porous and the center section is non-porous. This design allows tissue ingrowth into the ends but not the center of the stent-graft.

### EXAMPLE 5

### Method of Coating a Graft Using Solvents Having Different Boiling Points

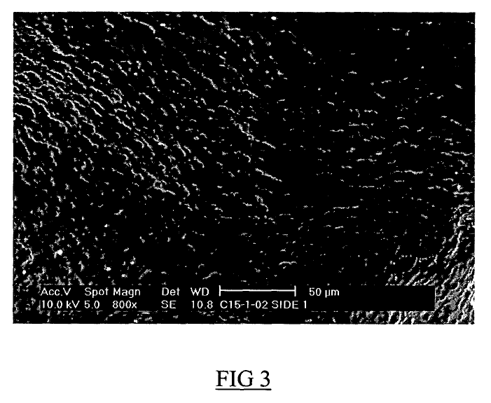

12 grams of SIBS are dissolved in 100 grams of hexane having a boiling point of 67° C. 40 grams of 2-butanol, having a boiling point of 98° C., is added to the solution. The solution is almost ready to precipitate but does not. A PET mesh is dipped into the solution, removed and placed in an oven at 70° C. The hexane then the 2-butanol are thereby flashed off causing the SIBS to precipitate and form a porous network that is well adhered to the PET mesh. A scanning electron micrograph of a coating formed in this manner is presented in FIG. 3.

### EXAMPLE 6

### Method of Coating a Graft Using Solvents Having Different Boiling Points

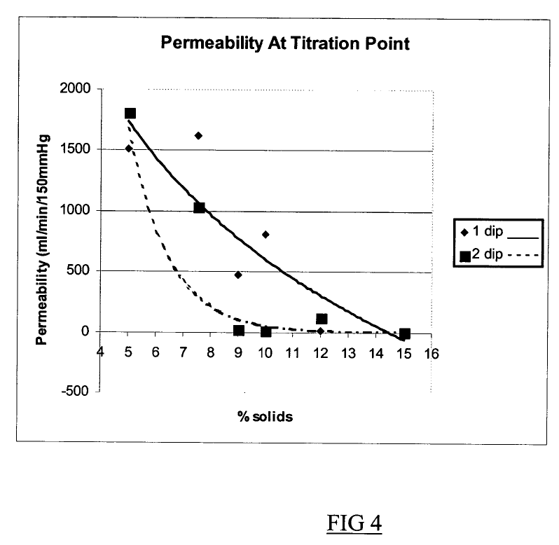

Different solutions ranging in solids content from 5% to 15% by weight of SIBS in hexanes are prepared. From the above solution, 10 mL is removed and combined with 4.5 mL of 2-butanol. The above mixture is quasi-stable, i.e. an additional few drops of 2-butanol will cause the SIBS to precipitate. This solution is now considered just below the titration point. FIG. 4 is a graph of solids content versus permeability of a PET mesh structure dipped into this solution and dried, thereby precipitating the copolymer. The first curve is for a single “dip” in the various solutions; the second curve is for two dips in the same solution. It can be observed that for a single dip, the permeability is zero at 15% solids, whereas for two dips, the permeability is zero at approximately 10% solids content.

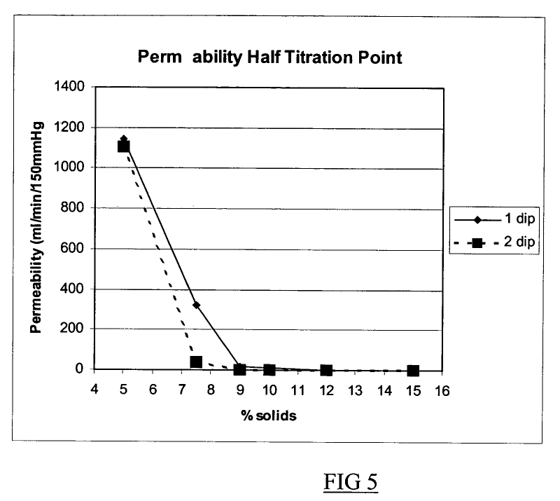

FIG. 5 presents curves similar to FIG. 4; however, in FIG. 5 the amount of 2-butanol is decreased to one-half of the titration point i.e. to one-half of the amount required to precipitate the solution. Accordingly, 2.25 mL of 2-butanol are used with the various solutions. FIG. 5 demonstrates that with one dip, a permeability of zero is achieved at approximately 9% solids, and that with two dips, a permeability of zero is achieved at 7.5% solids. It can be seen that by altering the solids content as well as the amount of the solvent in which the SIBS is not soluble, different permeabilities can be achieved.

### EXAMPLE 7

### Method of Coating a Graft Using Solvents Having Different Boiling Points

A vascular access graft is made as follows: A PET porous support structure, a scaffold, is sheathed over a mandril and the mandril with the PET scaffold is centered and fixed along the central axis of a metal tube. The annular space between the mandril and metal tube is filled with a solution of 12% SIBS in hexanes so that the solution resides on both sides of the PET scaffold as well as in the interstices of the scaffold. The assembly is then placed in a freezer at −10 C for 60 minutes thereby allowing the solution to gel and harden. The frozen structure is then pulled out of the tube and immediately placed in a container filled with 2-butanol. After soaking overnight, the phase inverted SIBS-coated scaffold is removed from the solvent and dried in a oven. A millimeter thick layer of porous SIBS is coated on both sides of the scaffold in this manner. This thick layer of SIBS allows the graft to be punctured with a large bore hypodermic needle and the needle removed without blood leaking from the needle site. This self-sealing characteristic of the graft is desirable for AV access grafts used in hemodialysis.

1. A method for the manufacture of a biocompatible porous prosthesis comprising the steps of:
(a) applying a solution to a porous support structure for a prosthesis, said solution comprising
(i) a biocompatible block copolymer including one or more elastomeric blocks and one or more thermoplastic blocks, and
(ii) a first solvent capable of dissolving said copolymer; and
(b) applying a second solvent capable of dissolving said first solvent but incapable of dissolving said copolymer to the surfaces of said prosthesis and thereby causing said copolymer to precipitate onto said support structure.

2. A method as recited in claim 1 wherein the porous support structure is a vascular stent, vascular graft, or vascular patch, a stent graft or a blood filter

3. A method as recited in claim 1 wherein the biocompatible block polymer has the general structure
(a) BAB or ABA,
(b) B(AB)ₙ or A(BA)ₙ, or
(c) X-(AB)ₙ or X-(BA)ₙ,
where A is an elastomeric block, B is a thermoplastic block, n is a positive whole number and X is a starting seed molecule.

4. A method as recited in claim 1 wherein the biocompatible block polymer is a triblock copolymer.

5. A method as recited in claim 1 wherein the block polymer is polystyrene-polyisobutylene-polystrene.

6. A method as recited in claim 1 wherein the solution of copolymer is applied to said support structure by dipping, submerging, solvent casting, spin coating, web coating, solvent spraying, ink jet printing or a combination of such processes.

7. A method as recited in claim 1 wherein said first solvent is a non-polar solvent.

8. A method as recited in claim 1 wherein said second solvent is a polar solvent.

9. A method as recited in claim 1 wherein said copolymer is present in said solution in from 0.5 to 50% by weight.

10. A method as recited in claim 1 wherein the coated porous support structure is heated or subjected to vacuum conditions to volatilize and thereby remove residual solvent.

11. A method as recited in claim 1 wherein the porosity of the copolymer deposited in the support structure is increased by reducing the concentration of copolymer in the solution.

12. A method as recited in claim 1 wherein the porosity of the copolymer deposited on the support structure is made greater, the greater the distance from the support structure, by one or more repetitions of steps (a) and (b), the concentration of copolymer in each sequential repetition of step (a) being less than in the prior step (a).

13. A method for the manufacture of a biocompatible porous prosthesis comprising the steps of:
(a) forming a solution comprising
(i) a biocompatible block copolymer comprising isobutylene and styrene or α-methylstyrene, and
(ii) a first, non-polar solvent selected from the group consisting of toluene, hexane, heptane, tetrahydrofuran, cyclohexane and methyl cyclohexane, capable of dissolving said copolymer, said solution comprising from 7% to 15% by weight copolymer,
(b) submerging a porous support structure for a prosthesis in the solution formed in step (a);
(c) removing the wetted support structure from said solution in step (b) and submerging it in a second, polar solvent selected from the group consisting of methanol, propanol, 2-propanol, ethanol, 1-butanol, 2-butanol, acetone and hexanol, capable of dissolving said first solvent but not capable of dissolving said copolymer, and thereby causing said copolymer to precipitate onto said support structure; and
(d) removing the coated support structure from said solvent in step (b) and removing residual first and second solvents from the coated support structure by volatilizing said solvents.

14. A method for the manufacture of a biocompatible porous prosthesis comprising the steps of:
(a) applying a solution to a mandril for a prosthesis, said solution comprising
(i) a biocompatible block copolymer including one or more elastomeric blocks and one or more thermoplastic blocks, and
(ii) a first solvent capable of dissolving said copolymer; and
(b) applying a second solvent capable of dissolving said first solvent but incapable of dissolving said copolymer to said mandril and thereby causing said copolymer to precipitate onto said mandril;
(c) removing solvent from the copolymer precipitated on said mandril by volatilizing it; and
(d) removing the so-formed prosthesis from said mandril.

15. A method for the manufacture of a biocompatible porous prosthesis comprising the steps of:
(a) applying a solution to a porous support structure for a prosthesis, said solution comprising
(i) a biocompatible block copolymer including one or more elastomeric blocks and one or more thermoplastic blocks, and
(ii) a mixture of solvents comprising a first solvent capable of dissolving said copolymer and a second solvent capable of dissolving said first solvent but not capable of dissolving said copolymer, said second solvent having a boiling point higher than that of said first solvent and being present in an amount less than that which causes said copolymer to precipitate out of said first solvent;
(b) volatilizing said first solvent from said solution, thereby causing said copolymer to precipitate onto said support structure.

16. A method as recited in claim 15 wherein solvent in the copolymer deposited on said support structure is removed by heating and/or subjecting the support structure to vacuum conditions.

17. A method as recited in claim 20 wherein said second solvent is present in said solution in an amount less than 95% of the titration point of said second solvent.

18. A method for the manufacture of a biocompatible porous prosthesis comprising the steps of:
(a) applying a solution to a mandril for a prosthesis, said solution comprising
(i) a biocompatible block copolymer including one or more elastomeric blocks and one or more thermoplastic blocks, and
(ii) a mixture of solvents comprising a first solvent capable of dissolving said copolymer and a second solvent capable of dissolving said first solvent but not capable of dissolving said copolymer, said second solvent having a boiling point higher than that of said first solvent and being present in an amount less than that which causes said copolymer to precipitate out of said first solvent;
(b) volatilizing said first solvent from said solution, thereby causing said copolymer to precipitate onto said mandril;
(c) removing solvent from said copolymer by heating said coated mandril; and
(d) removing said so-formed porous prosthesis from said mandril.

19. A method for the manufacture of a biocompatible porous prosthesis comprising the steps of:
(a) forming a solution comprising
(i) a biocompatible block copolymer comprising isobutylene and styrene or methylstyrene, and
(ii) a mixture of solvents comprising a first solvent capable of dissolving said copolymer and a second solvent capable of dissolving said first solvent but incapable of dissolving said copolymer, said second solvent having a boiling point higher than that of said first solvent and being present in an amount not exceeding 95% of that which causes said copolymer to precipitate out of said first solvent;
(b) submerging a porous support structure for a vascular prosthesis in the solution formed in step (a);
(c) removing the wetted support structure from the solution in step (b) and volatilizing said first solvent from the solution wetting said support structure, thereby causing said copolymer to precipitate onto the surfaces of said support structure; and
(d) removing said second solvent from the coated support structure by volatilizing it.

20. A porous prosthesis comprising a porous support structure coated with a biocompatible porous copolymer made by a process comprising the steps of:
(a) forming a solution comprising
(i) a biocompatible block copolymer comprising polystrene-polyisobutylene-polystyrene
(ii) a first solvent capable of dissolving said copolymer,
(b) submerging a porous vascular support structure for a vascular prosthesis in the solution formed in step (a);
(c) removing the wetted support structure from step (b) and submerging in a second solvent capable of dissolving said first solvent but not capable of dissolving said copolymer, and thereby causing said copolymer to precipitate onto the said support structure; and
(d) removing the coated support structure from the solvent in step (c) and removing said first and second solvents from the coated support structure by volatilizing them.

21. A porous prosthesis comprising polystrene-polyisobutylene-polystyrene made by a process comprising the steps of:
(a) forming a solution comprising
(i) a biocompatible block copolymer comprising polystrene-polyisobutylene-polystyrene
(ii) a mixture of solvents comprising a first solvent capable of dissolving said copolymer and a second solvent capable of dissolving said first solvent but not capable of dissolving said copolymer, said second solvent having a boiling point higher than said first solvent and being present in an amount not exceeding 95% of the titration point of said second solvent
(b) submerging a support structure for a prosthesis in the solution formed in step (a);
(c) removing the wetted porous support structure formed in step (b) from the solution and volatilizing said first solvent from the solution wetting said support structure, thereby causing said copolymer to precipitate onto said support structure; and
(d) removing second solvent from the deposited copolymer by heating the support structure,

22. A porous prosthesis comprising polystrene-polyisobutylene-polystyrene made by a process comprising the steps of:
(a) forming a solution comprising
(i) a biocompatible block copolymer comprising polystrene-polyisobutylene-polystyrene
(iii) a mixture of solvents comprising a first solvent capable of dissolving said copolymer and a second solvent capable of dissolving said first solvent but not capable of dissolving said copolymer, said second solvent having a boiling point higher than said first solvent and being present in an amount not exceeding 95% of the titration point of said second solvent
(b) submerging a mandril for a prosthesis in the solution formed in step (a);
(c) removing the wetted mandril formed in step (b) from the solution and volatilizing said first solvent from the solution wetting said mandril, thereby causing said copolymer to precipitate onto said mandril;
(d) removing second solvent from the deposited copolymer by heating the coated mandril, and
(e) removing the so-formed porous prosthesis from said mandril.

23. A method for the manufacture of a biocompatible porous prosthesis comprising the steps of:
(a) pouring a solution comprising:
(i) a biocompatible block copolymer including one or more a first solvent capable of dissolving said copolymer; into a mold;
(b) immersing said gel in a second solvent capable of dissolving said first solvent but incapable of dissolving said copolymer;
(c) heating the gel and second solvent and thereby causing the block, copolymer to precipitate and form a porous solid; and
(d) removing residual solvent from said porous solid by volatilizing it.

24. A method for the manufacture of a biocompatible porous prosthesis comprising the steps of:
(a) forming a solution comrpising
(i) a biocompatible block copolymer including one or more elastomeric blocks and one or more thermoplastic blocks, and
(ii) a first solvent capable of dissolving said copolymer;
(b) pouring said solution into a mold;
(c) chilling said solution to form a gel;
(d) removing said gel from said mold and immersing it in a second solvent capable of dissolving said first solvent but incapable of dissolving said copolymer;
(e) heating the gel and second solvent and thereby causing the block copolymer to precipitate and form a porous solid having interconnecting pores; and
(f) removing residual solvent from said porous solid by volatilizing it.

25. A method for the manufacture of a biocompatible porous prosthesis comprising the steps of:
(a) pouring a solution comprising
(i) a biocompatible block copolymer including one or more elastomeric blocks and one or more thermoplastic blocks, and
(ii) a mixture of solvents comprising a first solvent capable of dissolving said copolymer and a second solvent capable of dissolving said first solvent but not capable of dissolving said copolymer, said second solvent having a boiling point higher than that of said first solvent and being present in an amount less than that which causes said copolymer to precipitate out of said first solvent;
into a mold
(b) chilling said solution to form a gel;
(c) heating the gel to volatilize said first solvent and thereby cause the block polymer to precipitate and form a porous solid having interconnecting pores.

26. A method for the manufacture of a biocompatible porous prosthesis comprising the steps of:
(a) pouring a solution comprising
(i) a biocompatible block copolymer including one or more elastomeric blocks and one or more thermoplastic blocks, and
(ii) a mixture of solvents comprising a first solvent capable of dissolving said copolymer and a second solvent capable of dissolving said first solvent but not capable of dissolving said copolymer, said second solvent having a boiling point higher than that of said first solvent and being present in an amount less than that which causes said copolymer to precipitate out of said first solvent;
into a mold
(b) chilling said solution to form a gel;
(c) removing said gel from said mold;
(d) heating the gel and/or subjecting it to vacuum conditions to volatilize said first solvent and thereby cause the block polymer to precipitate and form a porous solid having interconnecting pores; and
(e) removing residual solvent from said porous solid.
